(19) United States
(12) Patent Application Publication
(10) Pub. No.: US 2004/0074499 A1
(43) Pub. Date: Apr. 22, 2004

(54) Tracheostomy tube and use of a coniostomy tube as a tracheostomy tube

(76) Inventor: Carl-Eric Lindholm, Uppsala (SE)

Correspondence Address: JACOBSON HOLMAN PLLC, 400 SEVENTH STREET N.W., SUITE 600, WASHINGTON, DC, 20004, US

(21) Appl. No.: 10/381,598

(22) PCT Filed: Sep. 20, 2001
(86) PCT No.: PCT/SE01/02016

(30) Foreign Application Priority Data
Oct. 5, 2000 (SE) 0003592-3

(51) Int. Cl. A61M 16/00

(52) U.S. Cl. 128/207.14

(57) ABSTRACT

A tracheostomy tube includes a tubular element that has a first straight section (62) which forms the patient-proximal end of the tube, a second straight section (61) which forms a machine-proximal end of said tube, and an intermediate curved section (63), wherein the two straight tube sections (61, 62) lie in a common plane. At least that part of the tube which passes through the opening made in the frontal wall of the trachea has a generally oval cross-sectional shape as viewed perpendicular to the long axis of the tube, wherein the minor axis of the oval cross-section is orientated generally in the curvature plane of the tube, and wherein the external ovality of the oval cross-section lies in the range of 1:12-1:1.40. The invention also relates to the use of a coniostomy tube of such design as a tracheostomy tube.

The invention relates to a tracheostomy tube of the kind defined in the preamble of claim 1.

The invention also relates to the use of a coniostomy tube as a tracheostomy tube.

It is well known to make an opening through the frontal wall of the larynx between the frontal part of the cricoid cartilage and the thyroid cartilage, and to insert into the opening a tube (a coniostomy tube) that is adapted to the insertion site. The advantage of coniostomy is that it is sometimes easier to open a passage to the respiratory tract by opening the cricothyroid membrane or ligament, which lies close to the skin, and to insert the coniostomy tube between the cricoid cartilage and the thyroid cartilage. One drawback with this is that the tube can cause an alteration to the patient's larynx and therewith affect the patient's vocal tones, even when the tube has an oval cross-section.

Alternatively, tracheostomy is carried out and an adapted tracheostomy tube is inserted into the resultant opening or stoma.

When a typical tracheostomy tube of circular cross-section and having the largest possible free area shall be inserted through the frontal wall of the trachea, it is, of course, necessary to make a sufficiently large opening through the skin and through said frontal wall. In addition to a membrane, the frontal part of the throat also includes a series of generally U-shaped and mutually parallel cartilaginous arches mutually spaced along the throat, the webs of said arches being situated in the frontal wall. The ends of respective legs of each arch are mutually joined via relatively soft tissue on the posterior side of the trachea.

In order to provide an opening which is large enough to allow a tube of circular cross-section to be inserted through the frontal wall of the larynx, it will normally be necessary to cut away or break of two or three mutually adjacent cartilage arches in the web region of the arch. The resultant damage to the throat may be relatively troublesome due to scar stenosis and also because the severed arches loose their arch function and are unable to hold stretched the membrane that bridges the arches, wherewith the whole of the segment of the trachea that was cut away may become soft and drawn inwards due to the fact that surrounding, undamaged arches are too widely spaced from one another to assist in holding the damaged part of the trachea in a stretched (open) state.

One object of the invention is to provide a technique for limiting the drawbacks that occur in respect of tracheostomy and in respect of the use of earlier known tracheostomy tubes.

This object is achieved with a tracheostomy tube according to the accompanying dependent claims.

The object of the invention is also achieved by using as a tracheostomy tube a coniostomy tube that is designed in accordance with the accompanying independent use claim.

Further embodiments of the invention will be apparent from the accompanying dependent claims.

The present invention means that fewer cartilage arches need be severed, or damaged in some other way, in order to permit insertion of the tracheostomy tube, therewith minimising damage to the throat, both in the long and the short term.

The present invention also means that the cartilage arch that lies closest to the stoma or opening in a direction towards the larynx will be subjected to a reduced pressure effect from the convex side of the curved part of the tube.

The invention will now be described by way of example, with reference to the accompanying drawings.

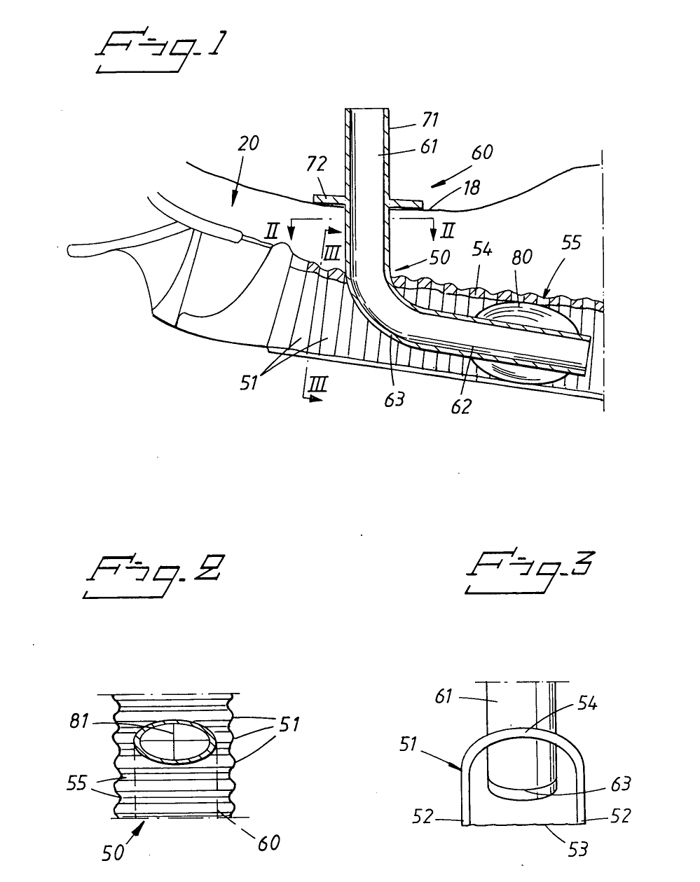

FIG. 1 is a schematic sectioned view of the neck region of a patient in which a tracheostomy tube has been inserted, said view being taken in the symmetry plane.

FIG. 2 is a schematic sectioned view taken on the line II-II in FIG. 1.

FIG. 3 is a schematic sectioned view taken on the line III-III in FIG. 1.

FIG. 1 illustrates schematically the larynx 20 of a patient and trachea or windpipe connecting with the larynx. The windpipe 50 includes a series of mutually parallel and mutually spaced generally U-shaped cartilage arches 51, shown in FIG. 3. The ends 52 of the legs of the arches are held together by soft tissue 53 on the distal side of the windpipe. The frontal side of the windpipe is defined by the webs 54 of respective U-shaped arches 51, and a membrane 55 which bridges the arches 51 and is held taut thereby.

A tracheostomy tube 60 includes a tubular element, which is either bent or bendable in a plane. As shown in the drawing, the tubular element has two legs 61, 62, which are mutually joined by an intermediate curved piece 63. A connecting stub 71 is found on the leg 61 of the tubular element, said connecting stub either being fixedly mounted or mounted for axial movement. The stub 71 has a flange 72 which abuts the frontal side 18 of the patient's neck. The leg 62 of the tubular element lies sealingly against the inner wall of the windpipe or trachea, through the medium of an inflatable balloon 80.

When the tube 60 is to be inserted in position, it is usual to surgically cut the soft parts of the neck to provide a relatively wide opening and to cut through the respective webs 54 of two (three) cartilage arches. Alternatively, a small incision may be made in the skin and an opening forcibly widened through the frontal side of the patient's neck, said opening also being extended through the membrane 55 and the arches 51. When the tube 60 has the requisite free cross-section and said cross-section is circular, it may be necessary to sever or break-off the web portions 54 of two or three cartilage arches in order to be able to insert the tube.

In accordance with the invention, the tracheostomy tube shall have an oval cross-section, as seen in FIG. 2, at least in that part of the tube which passes through the frontal wall of the trachea as defined by the cartilage arches. The minor axis of the oval cross-section lies in the curvature plane of the tube.

The outer ovality of the tube in the insertion area lies in the range 1:1.2-1:1.40, preferably 1:1.24-1:1.35. An ovality of about 1:1.24 is appropriate for males, while an ovality of about 1:1.27 is appropriate for females.

In respect of males, the external measurement of the tube in the insertion area is suitably 10.5-11.5 and 8.5-9.5 mm respectively (or 11-12 and 9-10 mm respectively when the wall thickness is 1.5 mm) along the major axis and the minor axis respectively of the oval cross-section. Correspondingly, in respect of women, the outer measurement is suitably 9.5-10.5 and. 7.5-8.5 mm respectively (or 10-11 and 8-9 mm respectively when the wall thickness is 1.5 mm). The tube will preferably have a wall thickness of about 1-1.5 mm. The tube of the illustrated embodiment has a wall thickness of about 1.25 mm.

The tube suitably has such an oval cross-section with the indicated orientation at least in the curve transition area, and the oval cross-section may also prevail along the full length of the tube. The inner angle between the tube section 61, 62 may, for instance, be 70-90°.

An inventive tracheostomy tube may also be used as a coniostomy tube.

The invention also relates to the use of a tube known from SE-B-465952 as a tracheostomy tube.

One advantage afforded by the oval cross-sectional shape of the inventive tube 60 in the region that passes the webs 54 of respective arches 51 resides in minimising the dimension of the tube in the longitudinal direction of the trachea such that probably only one single arch 51 need be severed or broken off at its web portion in order to enable the tube to be inserted. It will also be seen that the arch 51 lying closest above the tube 60 in the direction towards the larynx 20 is less affected by contact with the convex side of the curved oval tube 60. (In the case of a round tube, the cartilage is often pressed down and fractured so as to form a lip which projects into the windpipe and therewith contribute towards the formation of stenosis or stricture after the tube has been removed.) When only one cartilage arch is damaged, surrounding arches can, together, take over the arcade function of the damaged arch. Subsequent to having removed the tube 60 from the patient, scar stenosis is minimised at the insertion site. Neither will the membrane 55 sag inwards in a troublesome manner at the place of the arch 51 whose web 54 was destroyed in inserting the tube. It will be noted in particular that the arch 51 is no longer able to hold the soft tissue 53 taut after the web 54 of the arch 51 has been cut or broken away.

1. A tracheostomy tube for insertion into the trachea of a patient through an opening provided through a frontal wall of the trachea, said trachea including a plurality of mutually parallel and mutually spaced cartilage arches (51) which are mutually joined and covered by a membrane (55) and which have a generally U-shape and the ends (52) of said arches being mutually joined through the medium of soft tissue (53) on the dorsal side of the trachea (50), wherein the tube includes a tubular element which has an essentially straight section (62) which forms a patient-proximal end of the tube, an essentially straight tubular section (61) which connects with the machine-end of the tube (60) and which forms said machine end, and an intermediate curved section (63), wherein the two essentially straight tube sections (61, 63) lie generally in a common plane when the tube is inserted in place, characterised in that at least the part of the tube which passes through the opening in the frontal wall of the trachea has a generally oval cross-section, as seen perpendicular to the long axis of the tube, wherein the minor axis of the oval cross-section is orientated generally in the curvature plane of the tube, and wherein the external ovality of the oval cross-section is in the region of 1:1.2-1:1.40.

2. A tube according to claim 1, characterised in that the outer cross-sectional area in that part of the tube which is appropriate to said opening is chosen to essentially fill the area of said opening.

3. A tube according to claim 1 or 2, characterised in that the external ovality is in the range of 1:1.20 to 1:1.35.

4. A tube according to claim 3, characterised in that the external ovality is about 1:1.24 in the case of adult males and about 1:1.27 in the case of adult females.

5. A tube according to any one of claims 1-4, characterised in that the intermediate curved section (3) of the tube has an oval cross-sectional shape.

6. A tube according to claims 1-5, characterised in that the tube has an oval cross-sectional shape along at least the major part of its length.

7. The use of a tube which comprises a tubular element that has two essentially straight end sections (61, 62) and an intermediate curved section (63), wherein at least the part of the tube that shall extend through a frontal opening in a patient's trachea has a generally oval cross-sectional shape when seen perpendicular to the tube axis, with the minor axis of the oval tube cross-section orientated in the curvature plane of the tube and having an external ovality in the range of 1:1.2-1:1.40 in respect of said oval cross-section, as a tracheostomy tube.
